(19) United States
(12) Patent Application Publication
(10) Pub. No.: US 2018/0353542 A1
(43) Pub. Date: Dec. 13, 2018

(54) MONONUCLEAR-RICH, PLATELET-RICH PLASMA COMPOSITIONS AND METHODS OF USE THEREOF

(71) Applicant: Allan Kumar Mishra, Menlo Park, CA (US)

(72) Inventor: Allan Kumar Mishra, Menlo Park, CA (US)

(21) Appl. No.: 16/002,976

(22) Filed: Jun. 7, 2018

Related U.S. Application Data
(60) Provisional application No. 62/517,000, filed on Jun. 8, 2017.

(51) Int. Cl. A61K 35/15; A61K 35/19; A61K 35/16

(52) U.S. Cl. CPC A61K 35/15; A61K 35/16; A61K 35/19

(57) ABSTRACT

Compositions for platelet rich plasma (PRP) include a higher concentration of platelets and mononuclear cells than whole blood. Concentrations of lymphocytes, monocytes, and neutrophils can be different from concentrations found in whole blood. The compositions can be delivered at or near a site of tissue damaged by apoptosis, injury, wound, trauma, ischemia, lesion, or degeneration in order to treat the damaged tissue.

### CROSS-REFERENCE TO RELATED APPLICATIONS

This application claims priority to U.S. Provisional Patent Application No. 62/517,000, filed on Jun. 8, 2017, which is hereby incorporated by reference in its entirety and made part of this disclosure.

### FIELD OF THE INVENTION

The present application relates generally to formulations of a platelet rich plasma that can be used to treat various medical conditions. More specifically, the formulations of platelet rich plasma can comprise different levels of platelets and mononuclear cells relative to whole blood.

### BACKGROUND

Tissue damage and degeneration may be both a cause and an effect of various medical conditions. Causes of damage may range from mechanical injury to other complex physiological processes involving inflammation and the like. For example, tissue damage may be the result of injury, overuse, reduced blood flow, or any other suitable cause. Even if the damage is halted or slowed, the tissue may not completely heal due to the formation of degenerative, immature, avascular, and scar tissue.

Connective tissues, such as tendons, ligaments, joint capsules, fascial tissues, and the like may be especially prone to damage. The overall prevalence of musculoskeletal disorders, for example, is approximately 140 per 1000 persons in the United States, according to a 1995 survey by the National Center for Health Statistics. The same survey estimated the direct costs to be $88.7 billion and the indirect costs estimated to be up to $111.9 billion for lost productivity. Musculoskeletal injures may be resistive to standard treatments such as anti-inflammatory medication, bracing, rest and physical therapy. Injuries or other damage to flexible, relatively avascular connective tissues (hereafter “connective tissue” or “connective tissues”) may take a very long time to heal (e.g., months or even years). In many cases, injuries to connective tissues may never heal properly, and may require surgical intervention.

One example of a musculoskeletal disorder is lateral epicondylitis. Lateral epicondylitis or “tennis elbow” is a well-known sports medicine and orthopedic disorder that is often associated with to overuse injury and microtearing of the extensor carpi radialis brevis tendon at the elbow. The body attempts to repair these microtears but the healing process is incomplete in many cases. Pathologic specimens of patients undergoing surgery for chronic lateral epicondylitis reveal a disorganized angiofibroblastic dysplasia. This incomplete attempt at repair results in degenerative, immature, and avascular tissue. This incompletely repaired tissue may be weaker than normal tendon tissue and may lack normal function. This inadequate healing may continue to cause pain and may negatively impact the patient's ability to perform daily activities and the patient's quality of life.

Similar incomplete healing may be present in other types of musculoskeletal injuries or damage, such as patellar tendonitis (Jumper's Knee), Achilles tendonitis (common in runners), rotator cuff tendonitis (commonly seen in “overhead” athletes such as baseball pitchers), chronic injuries of the ankle ligaments (“ankle sprains”), or ligament tears.

Presently, many different non-operative and operative treatments exist. The non-operative measures include rest, activity modification, oral anti-inflammatory medication, and cortisone injections. While rest and activity modification may help patients with some of these conditions, there remains a significant clinical population that is not adequately treated with these therapies. Despite widespread use, oral anti-inflammatory medications have not proven to be useful in controlled studies. Some studies further suggest that non-steroidal medication may actually have an adverse effect on the healing process for ligament injuries. Also, no acute inflammatory cells have been found in pathologic samples of cases of lateral epicondylitis. Cortisone injections are controversial in the treatment of tendinoses and are contraindicated in acute ligament injuries. Several studies have noted an improvement in patients treated with cortisone in short term follow up, but longer term results beyond one year have revealed a high symptom recurrence rate and only an equivocal efficacy rate. These injections also carry the risk of tendon rupture, infection, skin depigmentation, subdermal atrophy, and hyperglycemia in diabetic patients. The operative measures include debridement and repair of the associated pathologic tendons. However, open or arthroscopic surgery has many potential complications such as deep infection, damage to neurovascular structures, and scar formation. The surgery is also expensive and carries the additional risks associated with regional or general anesthesia.

While musculoskeletal injuries may be associated with physical or mechanical processes, other types of tissue injury may involve physiological processes. For example, myocardial injury from a compromised cardiac vascular system may result in cell ischemia or even cell death. According to the American Heart Association, coronary heart disease is the single leading cause of death in the United States. The prevalence of heart attack in the U.S. was approximately 8.1 million people in 2005, and, of those, 920,000 were new or recurrent. Heart attack is also known as acute myocardial infarction (MI) and occurs when the blood supply to the heart is interrupted—usually by a plaque detaching from and blocking a cardiac blood vessel. As a result of restricted blood flow, the adjacent cardiac tissue becomes ischemic begins to die. If left untreated, an MI will lead to death.

Acute myocardial infarction may comprise non-ST-elevated myocardial infarction or ST-elevated myocardial infarction. In an ST-elevated myocardial infarction, the ST segment in an electrocardiogram (ECG) is elevated, meaning that the ventricles do not depolarize as rapidly as they would in a healthy heart. If blood flow to the heart is impaired over an extended period of time, an ischemic cascade and cardiac apoptosis may occur, causing heart cells to die and not regenerate. In place of the ischemic tissue, scar tissue forms. The scar tissue may increase the likelihood of cardiac arrhythmia, and may result in the formation of ventricular aneurysms.

To treat an MI, reperfusion therapy may be performed. Reperfusion therapies include thrombolytic therapy, percutaneous coronary intervention (PCI), and/or bypass surgery. While reperfusion therapy restores blood flow to the ischemic tissue, it does not lessen the risk of arrhythmia resulting from the growth of scar tissue. Because of the heightened risk of arrhythmia, the patient may be placed on anti-arrhythmia agents and/or require a pacemaker.

As such, additional treatments for treating tissue damage are desirable. Kits for treating tissue damage are also desirable.

### SUMMARY

In some aspects, a composition can comprise platelets in a concentration of about 151,000 per microliter to about 5,000,000 per microliter, mononuclear cells in a concentration about 500 to about 20,000 per microliter, and neutrophils in a concentration of about 1 to about 60,000 per microliter.

The composition of the preceding paragraph can also include any combination of the following features described in this paragraph, among others described herein. In some embodiments, the concentration of platelets can be between about 151,000 per microliter to about 5,000,000 per microliter. In some embodiments, the concentration of platelets can be between about 200,000 per microliter to about 2,000,000 per microliter. In some embodiments, the concentration of platelets can be between about 300,000 per microliter to about 1,500,000 per microliter. In some embodiments, the concentration of mononuclear cells can be between about 1,000 per microliter to about 15,000 per microliter. In some embodiments, the concentration of mononuclear cells can be between about 1,000 per microliter to about 20,000 per microliter. In some embodiments, the concentration of mononuclear cells can be between about 2,000 per microliter to about 20,000 per microliter. In some embodiments, the concentration of neutrophils can be between about 1,000 per microliter to about 10,000 per microliter. In some embodiments, the concentration of neutrophils can be between about 2,000 per microliter to about 5,000 per microliter. In some embodiments, the concentration of neutrophils can be less than about 2,000 per microliter. In some embodiments, the concentration of neutrophils can be less than about 5,000 per microliter. In some embodiments, the composition can further comprise red blood cells in a concentration of about 1 to about 10,000,000 red blood cells per microliter. In some embodiments, the composition can comprise less than about 4,000 red blood cells per microliter. In some embodiments, the composition can comprise less than about 1,000 red blood cells per microliter. In some embodiments, the composition can further comprise less than about 10,000 eosinophils per microliter. In some embodiments, the composition can be used to treat spinal disc injury. In some embodiments, the composition can be used to treat connective tissue injury. In some embodiments, the composition can be used to slow or stop cardiac apoptosis. In some embodiments, the composition does not include an exogenous activator.

Any of the features, components, or details of any of the arrangements or embodiments disclosed in this application are interchangeably combinable with any other features, components, or details of any of the arrangements or embodiments disclosed herein to form new arrangements and embodiments.

### DETAILED DESCRIPTION

Mononuclear-rich, platelet-rich plasma (MR-PRP) compositions are provided. The present compositions can generally include plasma that is enriched for platelets and mononuclear cells compared to their respective typical amounts in whole blood. The present compositions find use in treating damaged connective tissue, including spinal disc damage, and/or in treating ischemic tissue (e.g., after a myocardial infarction).

Outlined herein are compositions that include specific fractions of blood and that can be used alone, or in combination with other treatments, to treat a variety of injuries and disorders. Platelet rich plasma (PRP) can include a fraction of blood that contains a higher concentration of platelets than whole unconcentrated blood. A composition of the present disclosure can include platelet rich plasma and other blood fractions. In some embodiments, the present composition includes a fraction of blood that contains a higher concentration of mononuclear cells than whole unconcentrated blood. The peripheral blood mononuclear fraction can be defined as the summation of the monocytes (and/or macrophages) and lymphocytes. In some embodiments, this fraction is combined with platelets. Thus, a composition of the present disclosure can be a mononuclear-rich, platelet-rich plasma (MR-PRP) composition, in which the concentration of platelets and the concentration of mononuclear cells can be higher than their respective concentrations in whole blood. The MR-PRP compositions disclosed herein can provide a number of unexpected advantages, including any of increased efficacy, reduced side-effects, increased shelf-life, and other advantages over compositions having cell numbers outside the ranges disclosed herein.

In some embodiments, a MR-PRP composition includes about 151,000 platelets per microliter or more, e.g., about 200,000 platelets per microliter or more, about 300,000 platelets per microliter or more, about 400,000 platelets per microliter or more, about 500,000 platelets per microliter or more, about 750,000 platelets per microliter or more, about 1,000,000 platelets per microliter or more, about 2,000,000 platelets per microliter or more, about 5,000,000 platelets per microliter or more, including about 7,000,000 platelets per microliter or more, in combination with a mononuclear cell fraction in a concentration of about 500 cells per microliter or more, e.g., about 1,000 cells per microliter or more, about 2,000 cells per microliter or more, about 3,000 cells per microliter or more, about 5,000 cells per microliter or more, about 7,500 cells per microliter or more, about 10,000 cells per microliter or more, about 15,000 cells per microliter or more, including to about 20,000 cells per microliter or more. In some embodiments, the MR-PRP composition includes platelets in a range of about 151,000 to about 7 million platelets per microliter, e.g., about 151,000 to about 5 million platelets per microliter, about 175,000 to about 3 million platelets per microliter, about 200,000 to about 3 million platelets per microliter, about 200,000 to about 2 million platelets per microliter, about 500,000 to about 2 million platelets per microliter, about 700,000 to about 2 million platelets per microliter, about 300,000 to about 2,000,000 platelets per microliter, including about 300,000 to about 1,500,000 platelets per microliter, in combination with a mononuclear cell fraction in a concentration of about 500 to about 20,000 cells per microliter or more, e.g., about 750 to about 20,000 cells per microliter or more, about 1,000 to about 20,000 cells per microliter or more, about 1,000 to about 15,000 cells per microliter or more about, 1,500 to about 20,000 cells per microliter or more, including about 2,000 to about 20,000 cells per microliter or more. In some embodiments, the platelet concentration in the MR-PRP composition is in a range of about 200,000 to about 2 million platelets per microliter, in conjunction with a mononuclear cell fraction of about 1,000 to about 15,000 cells per microliter. In some embodiments, the platelet concentration in the MR-PRP composition is in a range of about 300,000 to about 1.5 million platelets per microliter, in conjunction with a mononuclear cell fraction of about 1,000 to about 20,000 cells per microliter. In some embodiments, the platelet concentration in the MR-PRP composition is in a range of about 500,000 to about 7 million platelets per microliter, in conjunction with a mononuclear cell fraction of about 2,000 to about 20,000 cells per microliter.

In some embodiments, a MR-PRP composition includes platelets at a concentration that is generally higher than the baseline, in combination with a mononuclear fraction that is higher than the baseline. Baseline concentration means the concentration found in the patient's blood which would be the same as the concentration found in a blood sample from that patient without manipulation of the sample by a laboratory technique, such as cell sorting, centrifugation or filtration. In some embodiments, the MR-PRP composition includes platelets at a concentration of 1.5 to 8 times the baseline or more, in combination with a mononuclear fraction of 1.5 to 8 times baseline or more. In some embodiments, the MR-PRP composition includes platelets at a concentration of 1.1 to 8 times the baseline, in combination with a mononuclear fraction of 1.1 to 8 times baseline. In some embodiments, the MR-PRP composition includes platelets at a concentration of 1.5 to 8 times the baseline, in combination with a mononuclear fraction of 1.5 to 8 times baseline.

The MR-PRP composition generally includes platelets at a concentration that is higher than the baseline concentration of the platelets in whole blood. The platelet concentration can be between about 1.1 and about 1.5 times the baseline, about 1.5 to 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration can be between about 4 and about 6 times the baseline. Typically, a microliter of whole blood includes at least 140,000 to 150,000 platelets and up to 400,000 to 500,000 platelets. The MR-PRP compositions can include about 500,000 to about 7,000,000 platelets per microliter. In some instances, the MR-PRP compositions includes about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter.

The MR-PRP composition can include a mononuclear fraction (e.g., lymphocytes plus monocytes) at a concentration that is higher than the baseline concentration of the mononuclear fraction in whole blood. The mononuclear cell fraction in the MR-PRP composition can be between about 1.1 and about 1.5 times the baseline, about 1.5 and about 2 times baseline, about 2 and about 4 times baseline, about 4 and about 6 times baseline, about 6 and about 8 times baseline, or higher. In whole blood, the mononuclear fraction is typically between 1,500 and 4,800 cells per microliter, but in other examples, the mononuclear fraction in whole blood is between 500 and 6,500 cells per microliter, or more.

In some embodiments, the MR-PRP composition includes a mononuclear fraction of from about 500 to about 1,000, about 1,000 to about 1,500, about 1,500 to about 2,000, about 2,000 to about 5,000, about 5,000 to about 10,000, about 10,000 to about 15,000, about 15,000 to about 20,000, or about 20,000 to about 25,000 cells per microliter or more.

In some embodiments, a MR-PRP composition includes, without limitation, neutrophils, red blood cells, basophils, eosinophils, and other blood cell fractions. The MR-PRP composition can include any suitable amount of neutrophils. In some embodiments, the MR-PRP composition includes substantially no neutrophils, or a concentration of neutrophils that is lower than the baseline concentration of neutrophils in whole blood. In some embodiments, the MR-PRP composition includes neutrophils at about the same concentration as the baseline concentration of neutrophils in whole blood. In some embodiments, the MR-PRP composition includes a higher concentration of neutrophils than the baseline concentration in whole blood. The neutrophil concentration can vary between less than the baseline concentration of neutrophils to eight times the baseline concentration of neutrophils. In some variations, the neutrophil concentration can be between about 0.01 and about 0.1 times baseline, about 0.1 and about 0.5 times baseline, about 0.5 and about 0.75 times baseline, about 0.75 times baseline to about 1.0 times baseline, about 1.0 and about 2 times baseline, about 2 and about 4 times baseline, about 4 and about 6 times baseline, about 6 and about 8 times baseline, or higher. The neutrophil concentration can additionally or alternatively be specified relative to the concentration of the lymphocytes and/or the monocytes. In some embodiments, the neutrophil concentration in the MR-PRP composition can be less than 0.75 times baseline. One microliter of whole blood typically comprises 2,000 to 7,500 neutrophils. In some variations, the PRP composition can comprise neutrophils at a concentration of about 1 to about 10,000 per microliter or more than about 10,000 per microliter. In some variations, the PRP composition can comprise neutrophils at a concentration of about 1 to about 1,000 per microliter, about 1,000 to about 2,000 per microliter, about 2,000 to about 5,000 per microliter, about 5,000 to about 10,000 per microliter, about 10,000 to about 20,000 per microliter, about 20,000 to about 40,000 per microliter, or about 40,000 to about 60,000 per microliter. In some embodiments, neutrophils are eliminated or substantially eliminated. Methods to deplete blood products, such as PRP, of neutrophils are known, as discussed in U.S. Pat. No. 7,462,268, filed on Aug. 16, 2005 and issued on Dec. 9, 2008, entitled PARTICLE/CELL SEPARATION DEVICE AND COMPOSITIONS, which is hereby incorporated by reference herein in its entirety.

In some embodiments, a MR-PRP composition includes red blood cells (RBCs). The MR-PRP composition can include any suitable amount of RBCs. In some embodiments, the MR-PRP composition includes substantially no RBCs, or a concentration of RBCs and/or hemoglobin that is lower than the baseline concentration in whole blood.

The RBC concentration can be between about 0.01 and about 0.1 times baseline, about 0.1 and about 0.25 times baseline, about 0.25 and about 0.5 times baseline, or about 0.5 and about 0.75 times baseline, about 0.75 times to about 0.9 times baseline. The hemoglobin concentration can be depressed and in some variations can be about 1 g/dl or less, between about 1 g/dl and about 5 g/dl, about 5 g/dl and about 10 g/dl, about 10 g/dl and about 15 g/dl, or about 15 g/dl and about 20 g/dl.

Typically, whole blood drawn from a male patient can have an RBC count of at least 4,300,000 to 4,500,000 and up to 5,900,000 to 6,200,000 per microliter while whole blood from a female patient can have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women.

In some embodiments, the MR-PRP composition includes RBCs in a concentration of from 1 to about 10,000,000 cells per microliter or more than about 10,000,000 cells per microliter. In some embodiments, the MR-PRP composition includes RBCs in a concentration of from 1 to about 1,000 cells per microliter, about 1,000 to about 4,000 cells per microliter, about 4,000 to about 10,000 cells per microliter, about 10,000 to about 20,000 cells per microliter, about 20,000 to about 30,000 cells per microliter, about 30,000 to about 40,000 cells per microliter, about 50,000 to about 100,000 cells per microliter, about 100,000 to about 500,000 cells per microliter, about 500,000 to about 1,000,000 cells per microliter, about 1,000,000 to about 5,000,000 cells per microliter, or about 5,000,000 to about 10,000,000 cells per microliter or more.

In some embodiments, the present MR-PRP composition includes the blood cell fractions as shown in Tables 1-5:

TABLE 1
MR-PRP preparation example 1

| Blood fraction | Concentration relative to baseline |
|---|---|
| Platelets | 5× |
| Mononuclear Cells | 5× |
| Neutrophils | Low/None |
| Red Blood Cells | Low/None |

TABLE 2
MR-RPR preparation example 2

| Blood fraction | Concentration relative to baseline |
|---|---|
| Platelets | 5.73× |
| Mononuclear Cells | 5× |
| Neutrophils | 0.452× (55% reduction) |
| Red Blood Cells | 0.056× (94% reduction) |

TABLE 3
MR-RPR preparation example 3

| Blood fraction | Concentration (per microliter) |
|---|---|
| Platelets | 300,000-1,500,000 |
| MononuclearCells | 1,000-20,000 |
| Neutrophils | 0-5,000 |
| Red Blood Cells | 0-1,000 |

TABLE 4
MR-RPR preparation example 4

| Blood fraction | Concentration relative to baseline |
|---|---|
| Platelets | 1.5×-8× |
| Mononuclear Cells | 1.5×-8× |
| Neutrophils | 0.75× or less |
| Red Blood Cells | 0.75× or less |

TABLE 5
MR-RPR preparation example 5

| | Concentration relative to baseline/ |
|---|---|
| Blood fraction | Concentration (per microliter) |
| Platelets | 1.1×-8×/500,000-7,000,000 |
| Mononuclear Cells | 1.1×-8×/2,000-20,000 |
| Neutrophils | 0× to 0.75×/0-5,000 |
| Red Blood Cells | 0×-0.75×/0-4,000 |

In some embodiments, the MR-PRP composition can include eosinophils and/or basophils. The eosinophils and/or basophils can be present in the MR-PRP composition at any suitable concentration. The concentrations of basophils and/or eosinophils in the MR-PRP composition can be less than baseline, about 1.5 times baseline, about 2 times baseline, about 3 times baseline, about 5 times baseline, or higher. In some embodiments, the basophils and/or eosinophils can be present in the composition in a concentration of 1 to about 10,000 cell per microliter or more than about 10,000 cells per microliter. In some embodiments, the basophils and/or eosinophils can be present in the composition in a concentration of 1 to about 10 cells per microliter, about 10 to about 30 cells per microliter, about 30 to about 50 cells per microliter, about 50 to about 100 cells per microliter, about 100 to about 1,000 cells per microliter, about 1,000 to about 2,000 cells per microliter, about 2,000 to about 5,000 cells per microliter, about 5,000 to about 10,000 cells per microliter or more. The eosinophil concentration can be between about 200 and about 1,000 per microliter elevated from about 40 to 400 in whole blood. In some variations, the eosinophil concentration can be less than about 200 per microliter or greater than about 1,000 per microliter.

The various blood fractions of the MR-PRP composition can be suspended in plasma or some other appropriate physiologically compatible fluid.

### Methods of Making

A composition of the present disclosure can include blood fractions (e.g., MR and/or PRP fractions) derived from a human or animal source of whole blood. The blood fractions can be prepared from an autologous source, an allogenic source, a single source, or a pooled source of platelets and/or plasma. To derive the blood fractions, whole blood can be collected, for example, using a blood collection syringe. The compositions of the present disclosure can be generated using any suitable method. The MR-PRP compositions are typically generated from whole blood or portions of whole blood using a variety of techniques comprising, for example, centrifugation, gravity filtration, and/or direct cell sorting. Once generated the MR-PRP compositions can undergo one or more processes to confirm the concentrations and/or activation of the various components. In some embodiments, the composition is generated using, without limitation, centrifugation, ficoll separation techniques, beads, columns, magnetic fields, cell sorting machines or other biologic, mechanical or electric means. The MR-PRP compositions can be generated by combining blood fractions containing the desired number of individual cell types.

### Methods of Use

Also provided herein are diagnostic or therapeutic methods of using a MR-PRP composition. A method of the present disclosure can include delivering a MR-PRP composition to an individual in need (e.g., a patient suffering from connective tissue damage, such as a spinal disc damage, from myocardial infarction (MI), and/or to slow or stop cardiac apoptosis after a heart attack). The MR-PRP composition can be used to treat any suitable disease or condition. In some embodiments, the method includes administering the MR-PRP composition to diagnose or treat a damaged connective tissue, such as, but not limited to, tendon, ligament, cartilage, spinal disc, muscle, bone, joint capsules, nerve, heart muscles, valves. or others. In some embodiments, the method includes using the MR-PRP composition to treat myocardial infarction. In some embodiments, the method includes using the MR-PRP composition to diagnose or treat acute or chronic spinal cord injury. Without being held to theory, it is thought that treatment with the present MR-PRP compositions can slow or stop apoptosis of myocardiocytes after a heart attack.

The MR-PRP composition can be used to diagnose or treat a variety of other conditions and disorders, including, but not limited to, hair loss (e.g., alopecia), skin conditions (e.g., for rejuvenation, wrinkles, acne, ulcerations, burns, etc.), eye-related disorders, ear conditions, internal organ dysfunction, erectile dysfunction, enhancement of female pelvic function (e.g., uterine or vaginal atrophy, or sexual dysfunction, etc.), osteoarthritis (e.g., of the ankle, knee, hip, sacroiliac joint, spine (including, but not limited to, facet joints), wrist, elbow, shoulder, etc.), low back pain and back muscle atrophy. In some embodiments, the MR-PRP composition can be used to diagnose or treat a neurodegenerative disorder or cancer.

To diagnose or treat tissue damaged by, for example, apoptosis, injury, wound, trauma, lesion, or degeneration, various methods for delivering the MR-PRP composition into the connective tissue and/or the myocardium can be employed. In various embodiments, the composition can be delivered to damaged connective tissues, the region of connective tissue directly adjacent to the damaged tissue, and/or healthy tissue. In some embodiments, the MR-PRP composition can be delivered to the ischemic tissues, the region of tissue directly adjacent to the ischemic tissue, and/or healthy tissue. The MR-PRP composition can comprise a platelet gel, or flowable material or liquid, other substances described herein, or any substance suitable for providing the desired level of treatment of the damaged or ischemic tissues.

The MR-PRP composition can be delivered to a patient in an emergency situation or as part of an elective procedure. To treat damaged connective tissue, the MR-PRP composition can be delivered as part of an inpatient or outpatient procedure days, weeks, months, or years after the tissue damage occurred. Examples of connective tissue damage that can be treated using MR-PRP include, but are not limited to, lateral epicondylitis (i.e., tennis elbow), plantar fasciitis, patellar tendonitis (i.e., Jumper's Knee), Achilles tendonitis, rotator cuff tendonitis, ankle sprains, and ligament tears. In some embodiments, the connective tissue damage is a spinal disc damage or injury. The tissue damage can be identified using one or more medical imaging technologies such as, but not limited to, x-ray imaging, magnetic resonance imaging (MRI), and ultrasound imaging. To treat damage to the myocardium, the MR-PRP composition can be delivered in an emergency room and/or by emergency medical service providers when an MI is identified. In other instances, the MR-PRP composition can be delivered after an MI during reperfusion therapy.

The MI can be identified by determining whether enzymes such as cardiac troponin (e.g., troponin-I or T), creatine kinase (CK) including CK-MB, aspartate transminase (AST)/Glutamic Oxaloacetic Transaminase (GOT/SGOT)/aspartate aminotransferase (ASAT), lactate dehydrogenase (LDH), and/or myoglobin (Mb), and/or the like are present in the blood stream. The MR-PRP compositions described herein can be delivered in the absence of the enzymes. Myocardial infarctions can be determined by identifying ST elevation in an ECG (e.g., during rest, a pharmacological stress test, and/or a physiological stress test), by coronary angiogram (e.g., noting acute closure of a vessel supplying myocardium at risk), by a nuclear medicine scan (e.g., technetium-99m or thallium-201), etc.

The MR-PRP composition can be delivered at any suitable dose. In some embodiments, the dose can be between about 1 cc and about 3 cc, between about 3 cc and about 5 cc, between about 5 cc and about 10 cc, between about 10 cc and about 20 cc, or more. The dose can be delivered according to a medical procedure (e.g., at specific points in a procedure) and/or according to a schedule. For example, prior to an elective cardioversion, the MR-PRP composition can be delivered about 24 hours, about 12 hours, about 6 hours, about 2 hours, and/or about 1 hour before the procedure begins. The MR-PRP composition is delivered to the patient at any suitable time of or after an injury. For example, treatment of a spinal cord injury can be performed at the time of the injury, or later.

In some examples, the MR-PRP composition can be delivered to a site of injury or damage (e.g., damaged connective tissue in or around affected joints or between affected vertebrae). The MR-PRP composition can be delivered to an individual in need thereof by injection using a syringe or catheter. In some embodiments, the MR-PRP composition can be delivered in conjunction with surgical intervention. The MR-PRP composition can also be delivered via a dermal patch, a spray device or in combination with an ointment, bone graft, or drug. It can further be used as a coating on suture, stents, screws, plates, or some other implantable medical device. Finally, it can be used in conjunction with a bioresorbable drug or device.

In alternate embodiments, a MR-PRP composition is incorporated into the device such as a suture, stents, screws, plates, or some other implantable medical device, during the manufacture of the device. The device in which a MR-PRP composition is already incorporated is then used for tissue repair.

In another embodiment, a MR-PRP composition is prepared and combined with a stent in an appropriate low oxygen chamber for 1-30 minutes, preferably about 10 minutes. The chamber is then exposed to ultraviolet light for a brief period of time, such as 1-60 seconds, 1-5 minutes, or 5-15 minutes. The stent is then removed from the chamber and implanted into a patient. It is expected that this chamber will improve the biologic activity of the MR-PRP composition and or device.

The site of delivery of the MR-PRP composition is typically at or near the site of tissue damage. The site of tissue damage is determined by well-established methods including imaging studies and patient feedback or a combination thereof. The preferred imaging study used can be determined based on the tissue type. Commonly used imaging methods include, but are not limited to, MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient can also assist in locating the site of tissue injury or damage by pointing out areas of particular pain and/or discomfort.

MR-PRP compositions that are formulated as gels or other viscous fluids can be difficult to deliver via a needle or syringe. Thus, in variations where the use of a needle or syringe is desirable, it can be desirable to add a gelling and/or hardening agent to the MR-PRP composition in situ. One or more needles or catheters can be configured to deliver the MR-PRP composition and/or the agent simultaneously, or substantially simultaneously, to the tissue. For example, if a needle is used to deliver the MR-PRP composition, the needle can comprise a plurality of lumens through which the MR-PRP composition and the agent separately travel. Alternatively or additionally, separate needles can be used to deliver the components to the tissue at the same time or one after the other.

The MR-PRP composition can be delivered minimally invasively and/or surgically. For example, the MR-PRP composition can be delivered using a catheter, e.g., which in some cases can be inserted into the patient via the femoral vein or artery, the internal jugular vein or artery, or any other suitable vein or artery. The MR-PRP composition can be delivered along with one or more medical devices, instruments, or agents to treat the MI, other cardiac conditions, spinal injury, and/or other tissue injuries or conditions, as described above.

The delivery system can deliver the components of the MR-PRP composition in a prescribed ratio (e.g., a ratio of the platelets and the mononuclear fraction). The prescribed ratio can be calculated beforehand or determined on an ad hoc basis once delivery begins. To deliver the components in the prescribed ratio, the delivery device can include one or more gears having a corresponding gear ratio, one or more lumens having a proportional lumen size, or any other suitable mechanism. Some delivery devices can include one or more mixing chambers. The multiple components can be delivered using separate delivery devices or can be delivered one after the other using the same delivery device.

The delivery devices can be advanced through a vessel adjacent to the tissue to be treated. The MR-PRP composition can be injected directly into the tissue using a needle and/or a needle-tip catheter. The MR-PRP composition can alternatively or additionally be infused into the vessel.

When the MR-PRP compositions are delivered using one or more catheters, any suitable catheter can be used. For example, the catheters can include one or more lumens and staggered or flush tips. The catheters can include needles or other devices (e.g., imaging devices) located at the distal end, and plungers or any other control located at the proximal end. The catheters and/or other delivery devices can have differently sized lumens to deliver multiple components of the MR-PRP composition in the prescribed ratio. When catheters are used, a physician can navigate to the heart using one of the routes known for accessing the heart through the vasculature, including but not limited to navigation to a heart chamber for epicardial, endocardial, and/or transvascular delivery of the MR-PRP composition.

The devices for injecting or delivering the MR-PRP compositions (catheter or otherwise) can include cooled parts or other temperature control mechanisms to keep the MR-PRP composition at a desired temperature. Various embodiments of delivery devices can include a cooled chamber, and/or an agitator mechanism in a MR-PRP chamber or injection chamber to prevent settling or clumping of the MR-PRP components. For example, in some variations, the catheter or other delivery device has a cooled lumen or lumens for keeping the MR-PRP composition cool during delivery. The delivery devices can additionally or alternatively include a mixing chamber for mixing the MR-PRP composition prior to delivery. The MR-PRP composition can also be stored in an agitating/vibrating chamber, or the physician can agitate the MR-PRP composition once inside the delivery device by tilting or otherwise manipulating the device.

A practitioner can make multiple deliveries into various locations using a single device, make multiple deliveries into various locations using multiple devices, make a single delivery to a single location using a single device, or make a single delivery to a single location using multiple devices. The delivery devices can include at least one reusable needle or catheter. Some embodiments can include delivery devices having an automated dosing system (e.g., a syringe advancing system). The automated dosing system can allow each dose to be pre-determined and dialed in (may be variable or fixed). In some embodiments, an iontophoresis device can be used to deliver the MR-PRP composition into the tissue.

The MR-PRP composition can alternatively or additionally be coated on one or more devices such as, for example, sutures, stents, screws, and/or plates. Anti-arrhythmia devices, such as pacemaker leads and automatic defibrillators can also be coated, sprayed, or dipped into the MR-PRP composition prior to, simultaneously with, or subsequently to implantation.

It can be desirable to deliver the MR-PRP composition to the ischemic tissues while avoiding coincidental delivery to other cardiac tissues or other locations adjacent to the heart. For example, the MR-PRP composition can gel or harden upon delivery to prevent migration. In some variations, a balloon catheter can be placed in the coronary sinus and inflated during delivery until the MR-PRP composition has solidified or at least partially immobilized. Other variations can include a pressure control system on the delivery device to prevent pressure-driven migration of the MR-PRP composition. Backbleed can also be prevented by keeping the needle in place for several seconds (e.g., about 5 to about 30 seconds, or about 5 to about 120 seconds) following an injection.

Sensors can be used to direct the delivery device to a desired location and/or to deliver the MR-PRP composition. For example, real-time recording of electrical activity (e.g., an ECG), pH, oxygenation, metabolites such as lactic acid, CO₂, or the like can be used. The sensors can be one or more electrical sensors, fiber optic sensors, chemical sensors, imaging sensors, structural sensors, and/or proximity sensors that measure conductance. The sensors can be incorporated into the delivery device or be separate from the delivery device. In some embodiments, the sensors can sense and/or monitor such things as needle insertion depth, blood gas, blood pressure or flow, hemocrit, light, temperature, vibration, voltage, electric current, power, and/or impedance. The sensors can include one or more imaging systems and can be coupled to any appropriate output device, for example, a LCD or CRT monitor which receives and displays information.

The MR-PRP composition can be used alone and or in combination with other therapies including, but not limited to, stem cells (embryonic, adult or iPS cells), cord blood, drugs, genetic engineering techniques, genetically engineered molecules, or other bioactive substances. Additionally, in some embodiments, the composition does not include an exogenous activator. Additional methods of use and embodiments for compositions are disclosed in U.S. patent application Ser. No. 13/333,082, filed on Dec. 21, 2011, now U.S. Pat. No. 8,444,969, issued on May 21, 2013, entitled NEUTROPHIL-DEPLETED PLATELET RICH PLASMA FORMULATIONS FOR CARDIAC TREATMENTS, which is hereby incorporated by reference herein in its entirety.

All of the features disclosed in this specification (including any accompanying exhibits, claims, abstract and drawings), and/or all of the steps of any method or process so disclosed, can be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Various modifications to the implementations described in this disclosure can be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

What is claimed is:

1. A composition comprising:
platelets in a concentration of about 151,000 per microliter to about 7,000,000 per microliter;
mononuclear cells in a concentration about 500 per microliter to about 20,000 per microliter; and
neutrophils in a concentration of about 1 per microliter to about 60,000 per microliter.

2. The composition of claim 1, wherein the concentration of platelets is between about 151,000 per microliter to about 5,000,000 per microliter.

3. The composition of claim 1, wherein the concentration of platelets is between about 200,000 per microliter to about 2,000,000 per microliter.

4. The composition of claim 1, wherein the concentration of platelets is between about 300,000 per microliter to about 1,500,000 per microliter.

5. The composition of claim 1, wherein the concentration of mononuclear cells is between about 1,000 per microliter to about 15,000 per microliter.

6. The composition of claim 1, wherein the concentration of mononuclear cells is between about 1,000 per microliter to about 20,000 per microliter.

7. The composition of claim 1, wherein the concentration of mononuclear cells is between about 2,000 per microliter to about 20,000 per microliter.

8. The composition of claim 1, wherein the concentration of neutrophils is between about 1,000 per microliter to about 10,000 per microliter.

9. The composition of claim 1, wherein the concentration of neutrophils is between about 2,000 per microliter to about 5,000 per microliter.

10. The composition of claim 1, wherein the concentration of neutrophils is less than about 2,000 per microliter.

11. The composition of claim 1, wherein the concentration of neutrophils is less than about 5,000 per microliter.

12. The composition of claim 1, wherein the composition further comprises red blood cells in a concentration of about 1 to about 10,000,000 red blood cells per microliter.

13. The composition of claim 12, wherein the composition comprises less than about 4,000 red blood cells per microliter.

14. The composition of claim 12, wherein the composition comprises less than about 1,000 red blood cells per microliter.

15. The composition of claim 1, wherein the composition further comprises less than about 10,000 eosinophils per microliter.

16. The composition of claim 1, wherein the composition is used to treat spinal disc injury.

17. The composition of claim 1, wherein the composition is used to treat connective tissue injury.

18. The composition of claim 1, wherein the composition is used to slow or stop cardiac apoptosis.

19. The composition of claim 1, wherein the composition does not include an exogenous activator.

20. A method of treating tissue damaged by apoptosis, injury, wound, trauma, ischemia, lesion, or degeneration, comprising delivering the composition according to claim 1 at or near a site of the tissue.
